(19) United States
(12) Patent Application Publication
(10) Pub. No.: US 2021/0030351 A1
(43) Pub. Date: Feb. 4, 2021

(54) METHOD, DEVICE AND PROGRAM FOR DETERMINING AT LEAST ONE DISTRIBUTION RATIO REPRESENTING CARRYING OUT A GIVEN PROCESS

(71) Applicants: UNIVERSITE DE RENNES 1, Rennes Cedex (FR); INSTITUT NATIONAL DE LA SANTE ET DE LA RECHERCHE MEDICALE, Paris (FR)

(72) Inventors: Mahmound Hassan, RENNES (FR); Fabrice Wendling, RENNES (FR); Aya Kabbara, RENNES (FR)

(21) Appl. No.: 16/966,745

(22) PCT Filed: Feb. 1, 2019
(86) PCT No.: PCT/EP2019/052549

§ 371 Date: Jul. 31, 2020

(30) Foreign Application Priority Data
Feb. 2, 2018 (EP) 18155011.2
Sep. 28, 2018 (EP) 18197414.8

(51) Int. Cl. A61B 5/00; G16H 50/30; G16H 50/70; G16H 40/67; A61B 5/0476

(52) U.S. Cl. CPC A61B 5/4094; G16H 50/30; G16H 20/30; G16H 40/67; A61B 5/0476; G16H 50/70

(57) ABSTRACT

A method of constructing a value representative of a local epileptogenic network index (LENI). The method is implemented by an electronic device that includes a processor and a memory. The method includes: obtaining, in the form of connectivity matrices, dynamic functional networks, which are representative of electrical signals measured for a predetermined number of points of interest, called nodes, within a cerebral cortex during a given time period; grouping the nodes, as a function of topological properties of said networks, within groups of nodes called modules; and calculating the local epileptogenic network index (LEND as a function of local functional connectivity characteristics of said nodes, modules and networks.

### 1. FIELD OF THE DISCLOSURE

The invention relates to brain networks characterization. More specifically, the invention relates to brain networks characterization in an epileptic context. Growing evidence suggests that alterations in large-scale networks are common substrate in a number of brain disorders, including epilepsies. Novel methods focusing on the estimation of brain connectivity from non-invasive data have emerged over the recent past years. Typically, several studies reported the potential ability of dense-Electroencephalography (EEG) source connectivity to estimate pathological networks at the cortical level from scalp EEG signals. An object of the invention is to propose a technique for identifying and quantifying epileptogenic networks from scalp EEG recordings.

### 2. BACKGROUND

The present section is intended to introduce the reader to various aspects of art, which may be related to various aspects of the present disclosure that are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Drug-resistant epilepsies, which represent 30% of epilepsies, are most often ‘partial’ or ‘focal’, i.e. characterized by an epileptogenic zone (EZ) that is relatively circumscribed in one of the two cerebral hemispheres. There is a large body of evidence supporting that neuronal networks in the EZ are characterized by an imbalance between excitatory and inhibitory processes which leads to increased excitability (Engel, 1996; Scharfman, 2007). This “hyperexcitability” which is the hallmark of epileptogenic networks is known to be at the origin of both interictal and ictal events. Resective surgery is currently the only treatment capable of suppressing drug-resistant seizures (ANAES, 2004). However, prior to surgery, the crucial issue to be solved is the identification of epileptogenic networks, in the specific context of each patient. Indeed, the outcome of this therapeutic approach directly depends on the capacity to accurately localize epileptogenic networks and subsequently define the optimal resection which maximizes benefit/deficit ratio for the patient, which is a serious issue.

Among pre-surgical investigations, stereoelectroencephalography (SEEG) represents, so far, the ‘gold standard’ for identifying epileptogenic networks and for accurately localizing the EZ (Bartolomei et al., 2017). Nevertheless, SEEG remains an invasive technique with limited spatial resolution. The demand is high for non-invasive, easy-to-use and clinically available methods able to reveal epileptogenic brain networks. To some extent, functional (fMRI, SPECT) neuroimaging methods (Schneider et al., 2013; Tavares et al., 2017), including electrical source imaging (ESI) (Michel et al., 1999; Lantz et al., 2001; Brodbeck et al., 2010; Lascano et al., 2012), are intended to respond to this demand. However, and despite the substantial progress accomplished in this field (Chiang et al., 2017), information provided by these techniques is not routinely used during pre-surgical evaluation due to intricate interpretation of localization results. There's thus a need for providing a non-invasive technique which allows identifying epileptic networks prior to examine the possibilities or decide of a possible resective surgery of networks.

### 3. SUMMARY

An object of the proposed technique is to process a neuromarker, based on EEG measurement, which allows defining a local epileptogenic network index.

According to an aspect of the present disclosure, these needs are at least partially fulfilled by a method of constructing a value representative of an interaction between a plurality of brain networks, the method being implemented by an electronic device, said electronic device comprising a processor and a memory. The method comprises:
obtaining, in the form of connectivity matrices, dynamic functional networks, which are representative of electrical signals measured for a predetermined number of points of interest, called nodes, within a cerebral cortex during a given time period;
grouping of the nodes, as a function of topological properties of said networks, within groups of nodes called modules;
calculating the index representing local epileptogenic network as a function of local functional connectivity characteristics of said nodes, modules and networks.

Thus, the method allows identifying epileptogenic networks with relatively simple data that can be computed from an EEG. Thus, the method allows obtaining information which may only be obtained by the use of surgery or complex implementation of expensive devices. Consequently, the proposed method is cheaper and less invasive than the previous ones, allowing to include these methods in standard routines.

According to a specific aspect, said local functional connectivity characteristics comprise: clustering coefficient, within-module degree and local efficiency.

According to a specific aspect, calculating said local epileptogenic network index comprises, for a given node i in a graph Gᵢ, comprising N nodes, connected to k edges, with a modular affiliation Mᵢ at a time period T, calculating:

$$LENI = \frac{2 {L}_{i}}{{k}_{i} ( {k}_{i} - 1 )} + \frac{{Z}_{i} ( {M}_{i} ) - \overline{{Z}_{i} ( {M}_{T} )}}{{σ}_{Z ( {m}_{i} )}} + \frac{1}{N} {\sum }_{i ∈ G} E ( {G}_{i} )$$

where
Lᵢ represents the number of links between the kᵢ neighbors of said node i;
σ denotes the standard deviation;
E is the local efficiency of a said node i represented by its Graph Gᵢ;
Gᵢ represents the graph of a given node i, i varying from 1 to N;
Zᵢ(Mᵢ) represents the number of edges connected to node i in module M.

Thus, the index is calculated for one or several nodes in graph Gᵢ. Optimizations can optionally be made on these calculations by trying to select nodes that are more likely to give an expected range of values. Such selection can for example be made on experience.

According to a specific embodiment, N is equal to 221. This number depends on various parameters, among which one can cite: the number of zones of the atlas, the number of electrodes which are used in the EEG.

According to an embodiment, obtaining connectivity matrices comprises:
obtaining signals representing of a cerebral activity for a given period of time;
constructing, using the previously obtained signals, a plurality of data structures representative of the functional connectivity between a plurality of regions of interest for a given frequency;
identifying, within said plurality of functional connectivity data structures, dynamic functional networks;

According to a specific feature, identifying comprises, for a group of functional connectivity data structures, implementing a method for detecting the dynamic community structure within said group of functional connectivity data structures.

According to an embodiment, grouping comprises:
determining topological properties of said dynamic functional networks previously obtained;
identification, as a function of said topological properties, of groups of nodes, called modules.

According to an embodiment, the disclosure also relates to an electronic device for obtaining a value representative of an interaction between a plurality of brain networks, the electronic device comprising a processor and a memory, characterized in that the device comprises the necessary means for:
obtaining, in the form of connectivity matrices, dynamic functional networks, which are representative of electrical signals measured for a predetermined number of points of interest, called nodes, within a cerebral cortex during a given time period;
grouping of the nodes, as a function of topological properties of said networks, within groups of nodes called modules;
calculating the index representing local epileptogenic network as a function of local functional connectivity characteristics of said nodes, modules and networks.

Of course, this electronic device comprises all the necessary means for implementing the proposed method. These means comprise computing resources, computing units, memory, databases access, networks interfaces, etc.

According to one specific implementation, the different steps of the method according to the invention are implemented by one or more software programs or computer programs comprising software instructions that are to be executed by a processor of an information-processing device, such as a terminal according to the invention and being designed to command the execution of the different steps of the methods.

The invention is therefore also aimed at providing a computer program, capable of being executed by a computer or by a data processor, this program comprising instructions to command the execution of the steps of a method as mentioned here above.

This program can use any programming language whatsoever and be in the form of source code, object code or intermediate code between source code and object code such as in a partially compiled form or in any other desirable form whatsoever.

The invention is also aimed at providing an information carrier readable by a data processor and comprising instructions of a program as mentioned here above.

The information carrier can be any entity or communications terminal whatsoever capable of storing the program. For example, the carrier can comprise a storage means such as a ROM, for example, a CD ROM or microelectronic circuit ROM or again a magnetic recording means, for example a floppy disk or a hard disk drive.

Furthermore, the information carrier can be a transmissible carrier such as an electrical or optical signal that can be conveyed via an electrical or optical cable, by radio or by other means. The program according to the proposed technique can especially be uploaded to an Internet type network.

As an alternative, the information carrier can be an integrated circuit into which the program is incorporated, the circuit being adapted to executing or to being used in the execution of the method in question.

According to one embodiment, the proposed technique is implemented by means of software and/or hardware components. In this respect, the term “module” can correspond in this document equally well to a software component and to a hardware component or to a set of hardware and software components.

A software component corresponds to one or more software module programs, one or more sub-programs of a program or more generally to any element of a program or a piece of software capable of implementing a function or a set of functions according to what is described here below for the module concerned. Such a software component is executed by a data processor of a physical entity (terminal, server, gateway, router etc.) and is capable of accessing hardware resources of this physical entity (memories, recording media, communications buses, input/output electronic boards, user interfaces etc.)

In the same way, a hardware component corresponds to any element of a hardware assembly capable of implementing a function or a set of functions according to what is described here below for the module concerned. It can be a programmable hardware component or a component with an integrated processor for the execution of software, for example, an integrated circuit, a smart card, a memory card, an electronic board for the execution of firmware etc.

Each component of the system described here above can of course implement its own software modules.

The different embodiments mentioned here above can be combined with one another to implement the proposed technique.

### 4. FIGURES

Explanations of the present disclosure can be better understood with reference to the following description and drawings, given by way of example and not limiting the scope of protection, and in which:

FIG. 1 describes the full pipeline of treatment of the data according to an embodiment;

FIG. 2 illustrates the mains steps of the process as disclosed;

FIG. 3 graphically represents an illustrative example of the analogy between the current understanding of the epileptogenic network and the graph theoretical measures adopted in the invention.

FIG. 4 is a representation of the results obtained by the method of the invention;

FIG. 5 disclose a simplified structure of a device of implementation of the process as disclosed.

### 5. DESCRIPTION

### 5.1. Principles

According to the invention, it is proposed a technique in which brain networks are built from dense-EEG recordings (the techniques for achieving this reconstruction of networks are known and are not part of the invention in itself). However, the source-space networks (obtained from EEG source connectivity method) are processed in a new and inventive way so as to provide an index called LENI: local epileptogenic network index which is based on the combination of several local functional connectivity characteristics (the clustering coefficient, the within-module degree and the local efficiency). For achieving these results, the inventors had the idea to use some mathematical tools, and more specifically some topological tools for mapping the functioning of the processing of the information in the brain with the ways the topological tools describe the functioning of networks.

The inventors used Dense-EEG (256 electrodes) connectivity at the source level for Epilepsy patients. The inventors confirmed that Epilepsy is a brain network disorder, characterized by an epileptogenic zone most often organized as a large-scale dysfunctional network involving multiple regions rather than a single focus. The inventor's technique support that EEG source connectivity complemented by graph theory leads to sparser networks which are more specific to epileptogenic networks, compared to the sole source localization approach. One explanation is that the source localization methods ignore the functional connectivity between brain regions, on one side, and ignore the possible contribution of brain sources with low energies, on the other side. In contrast, the network approach accounts for the communication dynamics between regions regardless of their energies.

The inventors also showed how local network measures may have a potential relation with the pathophysiology of epileptogenic networks. Indeed, based on the metrics introduced here (LEND, significant nodes correspond to pathological regions with high local connectivity. Indeed, both metrics quantify the implication of nodes within a local network and are able to localize the hemisphere and the lobe of stereo-EEG sites in the most patients. To emphasize that a good identification of epileptogenic network is related to the local properties of the network, the results obtained by other graph measures related to network global properties are also assessed: i) the betweenness centrality (C) which measures the importance of the node, and ii) the participation coefficient (P) which measures the global functionality of the node. Results showed that the identified regions using P and C global measures are distant from the SEEG contacts positions.

Thus, the method described here provides high value advantages (i.e. the non-invasiveness, minimal pre-processing of EEG signals recorded during resting state periods with no absolute necessity of including interictal epileptiform events), regarding previous existing techniques. The inventors believe that the proposed approach can bring relevant and complementary information in the context of pre-surgical evaluation. In particular, the additional clues provided by the method can be used by epileptologists in the definition of the best depth-electrode placement (hemisphere and lobe). In addition, due to the fact that SEEG cannot cover the whole surface of the brain in contrast to EEG, the proposed method may also highlight cortical regions that may be overlooked by the traditional pre-surgical evaluation.

The proposed method is included in the following general phases, some of which are more precisely described herein after:
data acquisition and preprocessing;
brain networks construction using the EEG source-connectivity method;
multi-slice networks modularity determination;
network measures for identifying local topological properties of the networks;
statistical tests;

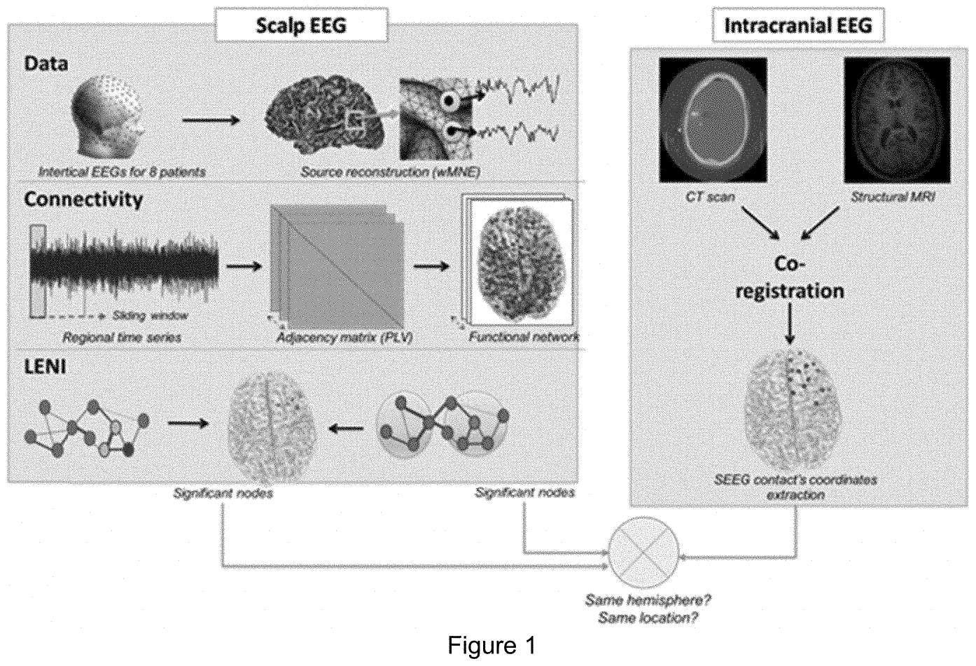

FIG. 1 illustrates the Structure of the process. On the left: the steps performed to identify the pathological nodes using EEG network analysis, for obtaining the LENI index. First, reconstruction of the regional time series using the weighted minimum norm estimate (wMNE) inverse solution. The dynamic functional connectivity matrices are then computed using a sliding window approach combined with the phase locking value (PLV) connectivity measure. After that, a combination of the within-degree module, the clustering coefficient and the local efficiency was used to quantify the local network property. Finally, the LENI is calculated. On the right, for comparison (and research) purposes of the identified networks: the step performed to extract the SEEG contacts' coordinates using the CT scan and the structural MRI images. Finally, the significant nodes obtained using EEG approaches are compared to the positions of SEEG contacts in terms of hemispherical, lobar (for demonstration of the method and research purposes).

According to the invention, once the networks are reconstructed from the EEG source connectivity method, these networks are characterized and the nodes which compose these networks are grouped together in modules (that is in set of nodes which are closely interconnected together while not being closely connected with other nodes of the networks). Each node is then characterized as a function of several measurements and calculations made on the connections of a node with other node. On the basis of the previous results combination of several local functional connectivity characteristics (the clustering coefficient, the within-module degree and the local efficiency) are calculated so as to provide a local epileptic network index.

More specifically, in relation with FIG. 2, it is disclosed a method of obtaining a value representative of an interaction between a plurality of brain networks, the method being implemented by an electronic device, said electronic device comprising a processor and a memory. The method (tested further on real brain data) comprises:
obtaining (10), in the form of connectivity matrices, dynamic functional networks, which are representative of electrical signals measured for a predetermined number of points of interest, called nodes, within a cerebral cortex during a given time period; this is obtained using the EEG source connectivity method implementation. The dynamic functional networks represent time-varying communications between a predefined number of regions of interests (221) in the brain.
grouping (20) of the nodes, as a function of topological properties of said networks, within groups of nodes called modules; it generally comprises grouping constituent regions of the previously detected functional networks, in the form of modules, a module corresponding to a set of intra-connected regions of interest (set of nodes), according to predefined grouping criteria. This grouping step (and its results) are used to compute the first local index called, the within-module degree.
calculating (30) the local epileptic network index, based on network measures obtained from grouped nodes within said modules (the within-module degree) combined with two other local measures: the clustering coefficient and the local efficiency. As explained herein after, the step of obtaining (10) connectivity matrices comprises:
obtaining (10-1) signals representing of a cerebral activity for a given period of time;
constructing (10-2), using the previously obtained signals, a plurality of data structures representative of the functional connectivity between a plurality of regions of interest for a given frequency;
identifying (10-3), within said plurality of functional connectivity data structures, dynamic functional networks;
As explained herein after, the step of grouping (20) comprises:
determining (20-1) topological properties of said dynamic functional networks previously obtained;
identification (20-2), as a function of said topological properties, of groups of nodes, called modules.

### 5.2. Description of an Embodiment

### 5.2.1. Materials and Methods

The full pipeline of the process is illustrated in FIG. 1, already presented.

### 5.2.1.1. Participants

This step is optional. The sole purpose is to obtain data which can be compared, for research and validation purposes. In total, eighteen patients with drug resistant epilepsy (18 males and 4 females, age 16-40 y) were included. These patients were diagnosed with drug resistant epilepsy. They underwent full presurgical evaluation including neurological examination, neuropsychological testing, standard long-term video EEG recording (32 electrodes, Micromed Inc.), structural MRI, dense scalp EEG recording (256 channels, EGI, Electrical Geodesic Inc.) with video recordings, CT scan and intracerebral EEG recordings (SEEG, Micromed Inc.).

### 5.2.1.2. Data Acquisition and Preprocessing

To evaluate the developed method, described above, dense EEG (256 electrodes) signals were recorded at 1000 Hz, band-pass filtered within 3-45 Hz, and segmented into three non-overlapping 40-seconds epochs. All epochs are chosen free of artifacts, during periods of quiet resting. For some patients, few electrodes with poor signal quality could be identified. For these electrodes, signals are reconstructed by interpolation of signals collected at the level of the surrounding electrodes.

For validation and research purposes (out of scope of the proposed method and index), SEEG recordings are performed using multi-contact intracerebral electrodes (10±18 leads; length, 2 mm, diameter, 0.8 mm; 1.5 mm apart) implanted according to Talairach's stereotactic method (Bancaud et al., 1970). The patient-specific positions of depth electrodes are determined by the neurological team, after detailed analysis of clinical, functional and anatomical data recorded for each patient. The exact 3D coordinates of each electrode contact are determined after co-registering the CT scan showing the intracerebral leads onto the structural MRI image using a 6-parameter rigid-body transformation (Studholme et al., 1998; Eickhoff et al., 2005).

### 5.2.1.3. Brain Networks Construction

The functional networks are reconstructed using the EEG source connectivity method, previously created by the inventors (Hassan et al., 2014). In brief, this method requires two main steps: i) solving the EEG inverse problem to reconstruct the temporal dynamics of the cortical regions at source level and ii) measuring the functional connectivity between the reconstructed regional time series. Here, the weighted Minimum Norm Estimate (wMNE) was used to reconstruct the dynamics of the cortical sources (Hamalainen and Ilmoniemi, 1994). Then, the functional connectivity was computed using the phase locking value (PLV) method (Lachaux et al., 1999). This combination of wMNE/PLV is proven to outperform procedures combining other inverse/connectivity methods, in terms of accuracy and relevance of cortical brain networks identified from scalp EEG data (Hassan et al., 2016, Hassan et al., 2014).

The steps performed to reconstruct the functional brain networks from dense-EEG signals can be summarized as follows, for a given patient:
Segment the T1-weighted anatomical MRI to build the cortical surface mesh using FreeSurfer (Fischl, 2012). This latter is then down-sampled into 15000 vertices using Brainstorm (Tadel et al., 2011).
Compute the lead field matrix using the boundary element method (BEM). Here, the inventors used the OpenMEEG package (Gramfort et al., 2010) available in Brainstorm.
The noise covariance matrix is estimated using one-minute resting segment.
Reconstruct the dynamics of EEG sources using the wMNE algorithm where the regularization parameter was set relatively to the signal to noise ratio (λ=0.1 in the actual proposed method).
Project the EEG sources onto an anatomical atlas. The inventors used the Desikan-Killiany atlas (Desikan et al., 2006) sub-divided into 221 regions as described in (Hagmann et al., 2008). The signals of the sources that belong to each ROI are averaged. This parcellation produced 221 regional time-series.
Compute the functional connectivity between the regional time-series using the PLV (Lachaux et al., 1999). This measure, ranging from 0 (no synchronization) to 1 (full synchronization), reflects synchrony. To explore the time dynamics of brain networks, the inventors used a sliding window over which PLV was calculated. Considering the investigated frequency range (0.3-45 Hz), the duration of the smallest time window that contains a sufficient number of cycles for PLV computation is 0.3 s. This value of 0.3 s was thus retained for the sliding window.
Threshold the connectivity matrix using the automatic thresholding algorithm described in (Genovese et al., 2002). According to this method, the connectivity matrix is converted into a p-value map based on the t-statistics. The computed p-values are corrected for multiple comparisons using the False Discovery Rate (FDR) approach of p<0.05. Then, the connectivity values whose p-values passed the statistical FDR threshold are retained (their values remained unchanged). Otherwise, the values were set to zero to build.

Consequently, at each time window, these steps produce a thresholded weighted connectivity matrix that is formally equivalent to an undirected weighted functional network. In the context of researches purposes, this method can be applied on several patients. However, in practice, this construction is performed for a single patient by the electronic device in charge of calculating the LENI index for this patient.

### 5.2.1.4. From Graph Theory to Epileptogenic Networks

According to the invention, once functional are reconstructed (in the form of weighted connectivity matrices), several transformation and calculation are made on these networks to obtain network characteristics which will allow calculating the LENI.

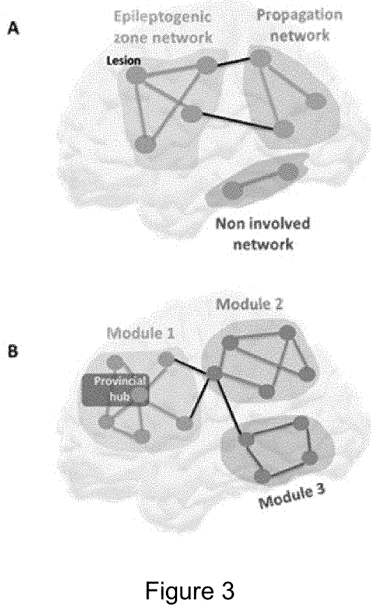

In this context, graph theory offers a framework to characterize the network topology and organization. In practice, many graph measures can be extracted from networks to characterize global and local network properties. Here, the inventors focused on measures quantifying the local connectivity of brain regions able to reveal sub-networks characterized by abnormal segregated neural processing. This choice was motivated by mechanistic hypotheses regarding the pathophysiology of epileptogenic networks. In particular, these “hyperexcitable networks” are likely characterized by abnormally high local “intra-connectivity” and weaker “inter-connectivity”. FIG. 3 illustrates the mapping which is produced by implementing the method of the inventors. (Up, FIG. 3, A) represents the organization of the epileptogenic networks in focal epilepsy (representation of a lambda patient): The epileptogenic zone (EZ) network contains brain regions (orange nodes) that may generate seizures. This EZ prompts another set of brain regions forming the propagation zone network (Green nodes). (Bottom, FIG. 3, B) represents the organization of the brain networks into modules, thanks to the use of the method according to the invention. Networks can be decomposed into modules. Edges are either linking nodes within modules (Orange, green or purple) or between modules (black edges). Highly connected nodes with other nodes in the same modules nodes are called provincial hub. In other words, based on the assumption, recently summarized in (Bernasconi, 2017) and illustrated in FIG. 3, A, the inventors hypothesized that an approach aimed at characterizing the local brain networks could be relevant for revealing hyperexcitable epileptogenic sub-networks in large-scale networks.

Following sections disclose the three network measures employed in respect to the mapping disclosed in FIG. 3.

Within-module degree: The modularity aims at decomposing a network into different communities of high intrinsic connectivity and low extrinsic connectivity. One of the metrics that can be extracted from the modularity-based analysis and describe the local functional connectivity is the within-module degree WMD, defined as:

$${WMD}_{i} = \frac{{Z}_{i} ( {M}_{i} ) - \overline{Z ( {M}_{i} )}}{{σ}_{Z ( {M}_{i} )}} \qquad ( 4 )$$

Where Zi(Mi) is the number of edges connected to node i in module M and σ is the standard deviation. A positive WMD value indicates that the node is highly connected to other members of the same community.

Average clustering coefficient: The clustering coefficient of a node represents how close its neighbors tend to cluster together. Accordingly, the average clustering coefficient of a network is considered as a direct measure of its segregation (i.e. the degree to which a network is organized into local specialized regions). In brief, the clustering coefficient of a node is defined as the proportion of connections among its neighbors, divided by the number of connections that could possibly exist between them.

Local Efficiency: The local efficiency of a network is the inverse of shortest path lengths. A short path length indicates that, on average, each node can reach other nodes with a path composed of only a few edges.

Statistical Tests

For a patient, one concatenated the distribution of the nodal metrics of the three epochs, which led to a distribution of (number of epochs×number of windows) values corresponding to each of the metrics extracted. To statistically identify the significant nodes in terms of local network measures, one quantified the difference between nodes metrics' distributions using a Wilcoxon Mann-Whitney U test. Thus, a 221×221 p-value matrix is generated, where the element p_{i,j} represents the statistical difference between the distributions of nodes i and j. The p-values are then corrected for multiple comparisons using Bonferroni correction method. Afterwards, the nodes that have a number of p-values above 99% of the confidence interval were considered as significant.

### 5.2.1.5. Calculation of the LENI Index

Based on the previous calculation, the network metric LENI (local epileptogenic network index) is calculated. This metric is based on the combination of several local functional connectivity characteristics (the clustering coefficient, the within-module degree and the local efficiency).

For a given node i (brain region) in a graph G (with N nodes) connected to k edges, with a modular affiliation Mi at period T (computed using Louvain algorithm), the new metric is defined as:

$$LENI = \frac{2 {L}_{i}}{{k}_{i} ( {k}_{i} - 1 )} + \frac{{Z}_{i} ( {M}_{i} ) - \overline{{Z}_{i} ( {M}_{T} )}}{{σ}_{Z ( {m}_{i} )}} + \frac{1}{N} {\sum }_{i ∈ G} E ( {G}_{i} )$$

where Li represents the number of links between the ki neighbors of node i and σ denotes the standard deviation. The new measure was normalized with respect to random networks. Thus, for each time window, one generated 500 surrogate random networks derived from the original network by randomly reshuffling the edge weights. The normalized values are then computed by dividing the original values by the average values computed on the randomized graphs.

E is the ‘local efficiency’ of a brain region i represented by it's Graph Gi. Gi represents the graph of a given node i (brain region i) varying from 1 to N (N being, in this example equal to 221).

Zi(Mi) represents the number of edges connected to node i in module M.

### 5.2.2. Comparison of Invasive Data vs. Noninvasive Data (For Research Purposes)

This section aims at proving that the noninvasive method proposed by the inventors allows obtaining accurate results, mitigating the needs of intracerebral electrode for determining the position of epileptic zones.

For each patient, significant nodes, as obtained using the EEG source connectivity method described, above were compared to the position of intracerebral electrode contact positions, as defined by the epileptologist during the pre-surgical planning procedure. To proceed, the SEEG electrode contacts were first projected into the same atlas of 221 ROIs: to each intracerebral contact we assigned the closest ROI from the 221 regions of atlas. Based on this co-registration in the grey matter, the position of scalp-EEG based significant nodes could be compared to that of SEEG contacts. This comparison gives an overall indication of the matching between noninvasive and invasive recordings (same hemisphere, same lobe, same sub-lobar region).

The qualitative results were also quantified using several performance measures:
The average distance (mm) between SEEG nodes and EEG nodes. AD is defined as follows:

$$A D = \frac{{\sum }_{k} d ( {N}_{k} , {N}_{v} )}{M} k ∈ [ 1 , M ] ; v ∈ [ 1 , W ]$$

Where d (Nₖ, Nᵥ) is the euclidian distance between the node Nₖ detected by EEG method and the nearest SEEG contact Nᵥ. M denotes the total number of detected EEG nodes, and W denotes the total number of SEEG contacts.
The closeness accuracy (%) which is defined as:

$$C A = \frac{{\sum }_{k} {x}_{k}}{M} ; {x}_{k} = 1 - \frac{d ( {N}_{k} , {N}_{v} )}{\overline{d}}$$

where d̅ is the mean Euclidian distance between EEG and SEEG nodes.
The hemispherical accuracy (%) which represents the proportion of the EEG nodes detected in the same hemisphere with the SEEG contacts.
The lobar accuracy (%) which represents the proportion of the EEG nodes detected in the same lobe with the SEEG contacts.
The overall accuracy (%) defined as the arithmetic mean of the three above-described accuracy values (closeness, hemispherical and lobar).

### 5.2.3. Results

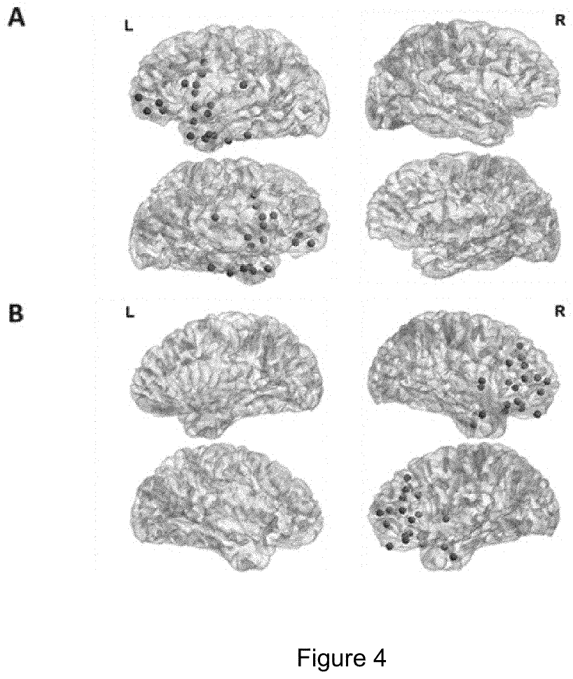

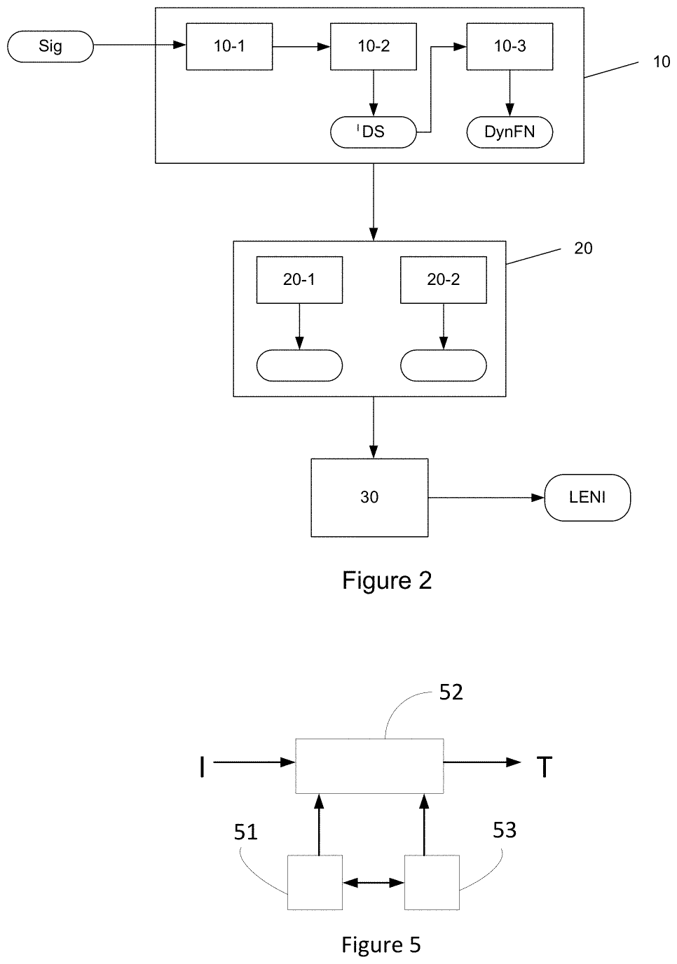

The cortical surface representations of the regions that showed high significant values (p<0.01, Bonferroni corrected) in local network measures (LENI) for two typical patients, are presented in FIG. 4. A node colored in blue represents a SEEG contact, not detected by EEG approach. A node detected in green represents a node detected by EEG approach. A node colored in red represents a node that coincides with a SEEG contact and is detected by EEG approach.

In FIG. 4, A, a first typical example of the LENI results is presented. The figure shows the matching between the nodes identified using LENI and the intracerebral EEG. An excellent hemispheric accuracy (100%) and lobar accuracy (100%) were observed with a distance=0 mm (all EEG-based nodes matched the SEEG nodes).

In FIG. 4, B, another example of the results using LENI is presented. The figure shows the matching between the nodes identified using LENI and the intracerebral EEG. An excellent hemispheric accuracy (100%) and lobar accuracy (100%) were observed with a distance of 18 mm.

### 5.2.4. Discussion

Identification of brain functional networks from scalp-EEG signals has been a topic of increasing interest over the two past decades (Hassan and Wendling, 2018). Emerging evidence shows the importance of identifying such networks at the cortical level (in the source space, in contrast with electrode space) using dense-EEG data (Hassan and Wendling, 2018). The approach, called “EEG source connectivity”, has led to novel findings regarding the spatio-temporal dynamics of functional brain networks, estimated from scalp-EEG data (Lu et al., 2012; Coito et al., 2015; M. Hassan et al., 2015). Here, we studied the applicability of network science applied to brain networks identified from non-invasive dense-EEG recordings at rest, in the aim of predicting stereo-EEG (SEEG) exploration in patients with refractory epilepsy, Inspired from the current understanding of epileptogenic networks characterized by hyperexcitability and hypersynchronization (review in (Bartolomei et al., 2017)), our approach was guided by the following hypothesis: can we identify sub-networks, referred to as “significant nodes”, characterized by significantly high local functionality while showing low interdependence level with large-scale networks at rest. Eventually, we substantiate the usefulness of our hypothesis by comparing the positions of nodes detected by scalp EEG to those of SEEG electrode. We found that the proposed approach has succeeded to identify significant nodes in the vicinity of the zone where SEEG implantation was performed. The major advantages of the presented approach are: i) the non-invasiveness of EEG, ii) the exploration of network dynamics at short time scale (hundreds of millisecond) and ii) the use of raw interictal recordings without pre-processing aimed at detecting epileptic events (like spikes or spike-waves). Results are discussed hereafter.

### 5.3. Devices and Computer Programs

Referring to FIG. 5, a simplified architecture of a device capable of implementing the proposed technique is described. Such a device comprises a memory 51, a processing unit 52 equipped for example with a microprocessor and driven by the computer program 53 implementing at least one part of the method as described. In at least one embodiment, the invention is implemented in the form of an application installed on a scheduling device. Such a device comprises the necessary means for implementing the proposed technique as described herein before. According to the disclosure, the device may be an independent device connected to an EEG recording and processing device or directly being integrated in an EEG recording and processing device.

1. A method comprising:
constructing a value representative of an interaction between a plurality of brain networks, the constructing being implemented by an electronic device, said electronic device comprising a processor and a memory, and the constructing comprising:
obtaining, in the form of connectivity matrices, dynamic functional networks, which are representative of electrical signals measured for a predetermined number of points of interest, called nodes, within a cerebral cortex during a given time period;
determining, from at least one of said connectivity matrices, a global efficiency (GE) score and at least one clustering coefficient score (Cc) for each node of said at least one of said connectivity matrices; and
calculating the value representative of an interaction between the plurality of brain networks in the form of a distribution ratio using said global efficiency score and said clustering coefficient scores (Cc), comprising for at least one connectivity matrix associated to at least one dynamic functional network:
$$DI = \frac{G E}{{\sum }_{i}^{N} C c}$$
where GE is the global efficiency of the network, Cc is the clustering coefficient and N is the number of nodes in the network.

2. The method according to claim 1, wherein determining said global efficiency (GE) score comprises calculating:
$$G E = \frac{1}{N} {\sum }_{i}^{N} {E}_{i}$$
where Eᵢ is the efficiency of each node I computed through the shortest path lengths between nodes.

3. The method according to claim 1, wherein determining one clustering coefficient score (Cc) of one node comprises calculating:
$$Cc ( i ) = \frac{2 {L}_{i}}{{k}_{i} ( {k}_{i} - 1 )}$$
where L represents the number of links between the kᵢ neighbors of node i.

4. (canceled)

5. The method according to claim 1, wherein N is equal to 68.

6. The method according to claim 1, wherein obtaining connectivity matrices comprises:
obtaining signals representing of a cerebral activity for a given period of time;
constructing, using the previously obtained signals, a plurality of data structures representative of the functional connectivity between a plurality of regions of interest for a given frequency; and
identifying, within said plurality of functional connectivity data structures, dynamic functional networks.

7. The method according to claim 1, wherein determining comprises, for a given connectivity matrix:
calculating the global efficiency (GE) score;
calculating an individual clustering coefficient score (Cc) for each node of said connectivity matrix.

8. An electronic device for obtaining a value representative of an interaction between a plurality of brain networks, the electronic device comprising:
a processor; and
a non-transitory computer-readable medium, comprising program code instructions which when executed by the processor configure the electronic device:
obtain, in the form of connectivity matrices, dynamic functional networks, which are representative of electrical signals measured for a predetermined number of points of interest, called nodes, within a cerebral cortex during a given time period;
determine, from at least one of said connectivity matrices, a global efficiency (GE) score and at least one clustering coefficient score (Cc) for each node of said at least one of said connectivity matrices; and
calculate the value representative of an interaction between the plurality of brain networks in the form of a distribution ratio using said global efficiency score and said clustering coefficient scores (Cc)), comprising for at least one connectivity matrix associated to at least one dynamic functional network:
$$DI = \frac{G E}{{\sum }_{i}^{N} C c}$$
where GE is the global efficiency of the network, Cc is the clustering coefficient and N is the number of nodes in the network.

9. A non-transitory computer-readable medium comprising program code instructions stored thereon which, when it is executed on a processor of an electronic device, configure the electronic device to obtain a value representative of an interaction between a plurality of brain networks by:
obtaining, in the form of connectivity matrices, dynamic functional networks, which are representative of electrical signals measured for a predetermined number of points of interest, called nodes, within a cerebral cortex during a given time period;
determining, from at least one of said connectivity matrices, a global efficiency (GE) score and at least one clustering coefficient score (Cc) for each node of said at least one of said connectivity matrices; and
calculating the value representative of an interaction between the plurality of brain networks in the form of a distribution ratio using said global efficiency score and said clustering coefficient scores (Cc)), comprising for at least one connectivity matrix associated to at least one dynamic functional network:
$$DI = \frac{G E}{{\sum }_{i}^{N} C c}$$
where GE is the global efficiency of the network, Cc is the clustering coefficient and N is the number of nodes in the network.
